(19) United States
(12) Patent Application Publication
(10) Pub. No.: US 2009/0005658 A1
(43) Pub. Date: Jan. 1, 2009

(54) Device for Determining the Physiological State of Female Mammals Related to Procreation

(75) Inventor: Rene Vinci, Perpignan (FR)

Correspondence Address: EGBERT LAW OFFICES, 412 MAIN STREET, 7TH FLOOR, HOUSTON, TX, 77002, US

(73) Assignees: Regine Vinci, Perpignan (FR); Arnaud Vinci, Perpignan (FR)

(21) Appl. No.: 12/160,103

(22) PCT Filed: Jan. 5, 2007
(86) PCT No.: PCT/FR2007/050615

§ 371 Date: Sep. 3, 2008

(30) Foreign Application Priority Data
Jan. 5, 2006 (FR) 06 00151

(51) Int. Cl. A61B 10/00

(52) U.S. Cl. 600/301; 600/551

(57) ABSTRACT

The invention relates to a device for determining the physiological state of female mammals related to procreation. The inventive device comprises a sealed housing (2) which is introducible into the reproductive tract of a female mammal and comprises at least two sensors (3) placed on the periphery thereof and/or therein for capturing different physical and/or psychological data items and a power supply means for enabling the device to be independently operational. Said housing (2) is also connected to means for processing data items collected by said sensor(s) (3) and to means for transmitting the collected data items and/or results obtained by data processing.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to a device for determining the physiological state of female mammals related to procreation.

The invention falls within the field of the determination of the physiological state of female mammals related to procreation.

2. Description of Related Art Including Information Disclosed Under 37 CFR 1.97 and 37 CFR 1.98.

The physiological state of female mammals related to procreation comprises namely important particular states, such as the fertile state, pregnancy or the imminence of the parturition or delivery.

In women, the fertile state corresponds to the period of ovulation during which she is likely to be fertilized. In female mammals in general, the fertile state corresponds to the period of heats.

Pregnancy intervenes as soon as a fecundation occurred, and is completed at the moment of parturition or delivery in women.

Whether in animals or human beings, it is very useful to be able to accurately determine the occurrence of such states, and their duration intervals. For the fertile state, this is interesting in particular with a view to an artificial insemination, in order to achieve a controlled fecundation. Indeed, the failures of artificial insemination remain numerous and the presence or not of a fertile state is one of the variables. These failures have in addition a high cost.

Physiologically, the entering into a period in which the ovule is fertilizable or about to be so, results into changes in temperature and hormone concentrations in the vagina of the female mammals.

Likewise, the study of these physiological parameters and their changes can permit detection of pregnancy, for example following a successful insemination, or the imminence of the parturition or delivery, which are equally interesting to be foreseen.

There are already known devices for assessing in particular the fertile state of a female mammal. The simplest ones of them use a manual probe for measuring the reproductive tract temperature; others measure the physical parameters of the vaginal mucous membrane by manually inserting a measuring probe.

First of all, these devices are not convenient in use and provide questionable results.

Moreover, they cannot be used in an automated way, in particular in large mammal farming.

### BRIEF SUMMARY OF THE INVENTION

The present invention pretends to cope with these two major disadvantages in the field of the determination of the physiological state of female mammals related to procreation.

In addition, the present invention applies both to women and female mammals in general.

To this end, the invention relates to a device for determining the physiological state of female mammals related to procreation. The device comprises a sealed housing aimed at being inserted into the reproductive tract of a female mammal and comprising, on the one hand, on its periphery and/or internally at least two sensors for capturing different physical and/or psychological data and, on the other hand, power supply means for enabling the device to be independently operational. The housing is associated with means for processing data collected by said sensor or sensors as well as means capable of transmitting the collected data and/or the results obtained by said data processing.

Advantageously, one of the sensors for capturing physical and/or physiological data of the housing consists of a temperature sensor.

One of the sensors for capturing physical and/or physiological data can also consist of a sensor permitting to determine the presence and/or the concentration of hormones.

The housing can in addition comprise means for diffusing a substance into the reproductive tract of the female mammal.

Thanks to the simultaneous examination of at least two physiological parameters, in particular the temperature and the hormone concentration in the vagina of a female mammal, the invention permits to accurately determine one of the physiological states related to procreation, such as a state of ovulation and the duration of the period of fertility and/or a state of pregnancy and/or the imminence of the parturition or delivery.

In addition, it advantageously permits to automatically and quickly use physical, physiological and biological data collected in situ in the reproductive tract of a female mammal.

Other features, aims and advantages of this invention will become clear from the following detailed description, with reference to the attached drawings referring to two exemplary embodiments given as an indication and in a non-restrictive way.

### BRIEF DESCRIPTION OF THE SEVERAL VIEWS OF THE DRAWINGS

FIG. 1 shows a schematic view of a particular embodiment of the device for detecting the fertile state according to the invention.

FIG. 2 shows a schematic view of an alternative of the embodiment of FIG. 1.

FIG. 3 shows another schematic view of an alternate embodiment of the device for detecting the fertile state according to the invention.

### DETAILED DESCRIPTION OF THE INVENTION

As can be seen in the attached drawings, the present invention relates to the field of the determination of the physiological state of female mammals related to procreation, and relates, in particular, to a device for determining the physiological state of female mammals related to procreation.

More specifically, the invention aims at permitting the detection of parameters for determining namely the fertile state and/or the pregnancy and/or the imminence of the parturition or delivery in female mammals.

Thanks to an examination over the time of accessible physiological parameters of the vagina, it is possible to determine the time at which the ovule is fertilizable or about to be so, which namely results into changes in temperature and hormone concentrations in the vagina.

In a similar way, it is possible to detect a pregnancy or the imminence of a parturition or delivery.

It is thus in particular the examination of these changes that permits to determine an entering into a fertile state, a pregnancy or the imminence of the parturition or delivery.

The invention is aimed at permitting to detect the values of some physiological parameters, namely in order to examine them over the time.

For greater clarity's sake, the following detailed description will mainly refer to the detection of the fertile state of female mammals. The transposition to the detection of pregnancy or imminence of a parturition or delivery is easy, the only difference residing in the analysis, eventually over the time, of the detected values and/or the selection of the parameters to be observed.

The preferred examination parameters are formed by temperature, directly accessible, and the hormone concentration, namely the progesterone concentration.

Indeed and in this respect, the ovulation process is accompanied by an increase of the rate of progesterone permitting to detect an ovulation and, hence, a fertile state.

Generally, it is of course possible to detect other hormones in the vagina of female mammals, which can be used to determine the physiological state of female mammals related to procreation.

The examination of the hormone concentration can be performed directly. In the case of progesterone, it can also be obtained by observing consecutive side effects to the changes in progesterone concentration. This consists for example in measuring the resistance or resistivity of the vaginal mucous membrane or the mucus, which is directly related to the progesterone concentration.

In particular, the dates or times corresponding to characteristic changes, such as a sudden rise or a sudden lowering, of the above-mentioned parameters inform on a fertile state.

Moreover, the invention takes advantage of a simultaneous use of the changes in at least two parameters, in particular the temperature and the hormone concentration, this use resulting into determining a state of ovulation or its imminence.

In order to examine the changes of these parameters, a device 1 is used that comprises at least a sealed housing 2 aimed at being inserted into the reproductive tract of a female mammal, said housing 2 including on its periphery and/or internally at least two different sensors 3. The housing 2 includes power supply means 4 permitting its independent operation, in particular in the form of a battery 4, and means for processing 5 the information provided by the sensors 3. Of course, the device 1 also comprises means capable of transmitting the collected data and/or the results obtained from said data processing.

The latter means capable of transmitting the collected data and/or the results obtained from said data processing allow reproducing the data collected and/or the results obtained from the data processing on any type of carrier, for example by means of a computer screen, a printer or also in the form of light or sound signals.

Preferably, the at least two types of sensors 3 used permit, on the one hand, to measure the temperature and, on the other hand, the hormone concentration. It is of course possible to use, in addition, sensors 3 with different functions, as will be explained below.

It will also be understood that the examination of the progesterone concentration is particularly useful, without the invention being limited thereto.

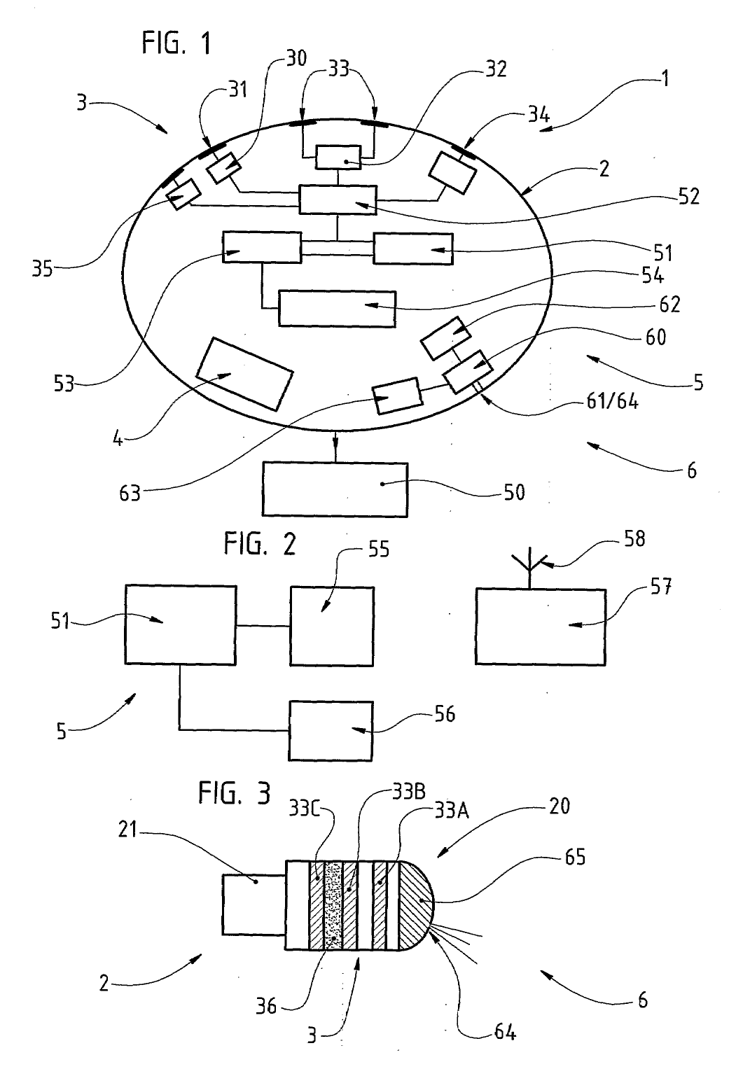

FIG. 1 shows a particular embodiment of the device according to the invention, comprising a temperature sensor 30 and its probe 31 to be placed in contact with the vaginal wall. One can also imagine using a temperature sensor fully insulated in the housing 2.

Various types of sensors can be contemplated, and this in particular for measuring the hormone concentration.

Thus, the change in hormone concentration is easily and preferably accessible by measuring the resistance or resistivity of the mucous membrane and/or mucus. The embodiment of FIG. 1 shows a housing 2 incorporating a sensor 32 sensitive to the resistance of the vaginal mucous membrane with at least 2 electrodes 33 to be placed in contact with the vaginal wall and/or mucus.

FIG. 3, which shows an embodiment of the housing 2 according to the invention particularly adapted for use in women due to its ovule-shape, also comprises a sensor 32 permitting to measure the resistance of the vaginal mucous membrane between the electrodes 33A and 33B and the resistance of the coating or mucus between the electrodes 33B and 33C.

The change in hormone concentration is also accessible through biosensors having molecular sites allowing fixing hormones, said fixing inducing a change in electric quantities.

Thus, the embodiment of FIG. 1 also shows a biosensor 34 sensitive to the estrogens and a biosensor 35 reacting to the pheromones secreted by the Bertholin glands.

As biosensor, there can be used a coating which, when getting impregnated with hormones, for example through absorption, undergoes a change in resistivity.

In the embodiment of FIG. 3, the housing 2 also comprises a sensor 36 in the form of a bioactive coating 36 capable of reacting with the organic molecules present in the vagina and the mucous membrane.

As explained above, these two resistances finally inform, by means of their measured values or their changes, on the presence and/or the evolution of the hormone concentration in the mucus and the vagina.

According to another advantage of the invention and in order to provide an automated solution for the detection of the fertile state in female mammals, means for processing 5 the information provided by the sensors 3 have been integrated into the device 1 according to the invention.

These processing means 5 are preferably distributed between the inside and the outside of the housing 2.

The measurements collected by the sensors 3 can thus be processed directly in the housing 2 and/or be transmitted by telemetry to an external central processing unit 50. The result of the data processing is of course aimed at determining the fertile or non-fertile state of the female mammal tested and/or determining a period of fertility.

In the same way, one can look for a pregnancy and/or the imminence of a parturition or delivery.

The direct processing of the measurements inside the housing 2 is performed by means of at least one memory 51 connected to a control unit 52, permitting to record, at regular intervals, for example the temperature, the resistance of the vaginal wall, the estrogen concentration or the presence of pheromones, these parameters depending on the sensors 3 present.

A central processing unit 53 located inside the housing 2 and including the memory and processing software then permits to ensure the analysis of these measurements collected by the sensors 3 and the sequencing of the measurement and recording operations.

The processing software permits namely to accurately determine a period of fertility.

Advantageously, once the analysis of these measurements has been performed, the result of the latter is transmitted by telemetry via a transmitting-receiving unit 54 ensuring the dialogue and the transfer of the results with the fixed central processing unit 50. The transmitting-receiving unit 54 advantageously comprises a transmitting-receiving module.

In this respect, the central processing unit 53 also manages the sequencing of the transmission towards the external central processing unit 50.

According to a variant of the device 1 according to the invention, the transfer of the measurements performed can also occur without previous analysis inside the housing 2, or in addition to the transfer of the analysis performed inside the housing 2.

According to the particular embodiment of FIG. 1, the measured values are recorded at fixed dates and hours and stored in the memory 51.

These values are then either used locally by the processing software of the central processing unit 53, or transmitted to the external central processing unit 50, for example, in the case of an application to herds of animals, at the time of passing of each animal in front of a reception arcade or at distances from the place of pasture.

As set forth above, various options are possible, such as the final transmission only of the analysis performed by the central processing unit 53, or of both this analysis and the measurements collected at the level of the sensors 3, or only the measurements collected at the level of sensors 3, which eventually also require a subsequent processing externally to the housing 2 in order to determine the state of fertility and/or pregnancy and/or the imminence of a parturition or delivery.

In this context, the result of the analysis, permitting to determine in particular a favorable period for fertilization, is then accessible to the persons in charge of the reproduction, who can decide to try an insemination or not.

Thus, the device of this invention permits to know with a good probability the periods of fertility of the females of a herd of mammalian livestock, limiting as well as possible the failures and the cost of the artificial insemination, or also keeping the males away during these same periods.

This is also very useful for preparing a possible veterinary intervention in the case of parturition.

FIG. 2 shows a variant of the device 1 according to FIG. 1, in which the data of the memory 51 are written in a RFID module 55 by a writing module 56.

Outside the housing 2, a module 57 for activating and reading the RFID with its antenna 58 permits to read the data contained in the RFID module 55 connected to the memory 51 and to process them. The module 57 also permits to write into said RFID module 55.

This variant, which uses the radio-identification, is particularly relevant for monitoring herds of animals. The following example refers to a herd of bovines. Each cow is permanently equipped with a housing 2, which permanently transmits, at regular intervals, an own digital code. Upon reception of this code, the fixed central processing unit 50, arranged in the shed, sends a signal to the housing 2 requesting same to transmit the background of the parameter values recorded in the form of curves. This background is received by the central processing unit 50 and processed to update one of the information “ovulation”, “pregnancy” or “imminence of a parturition” contained in the change of the recorded parameters, and this individually for each cow.

Advantageously, as shown in FIG. 1, the housing 2 of the device according to the invention also comprised means for diffusing 6 a substance into the reproductive tract of the female mammal in the form of at least one solenoid valve 60 capable of injecting, through a tube 61 and under control of a control unit 62, a substance contained in a pressurized tank 63. Said control unit 62 can, preferably, be under remote control, and thus be activated from outside the housing 2.

The control unit 62 can also be under the control of the central processing unit 53.

In either case, it can also be activated in an automated way, depending on the values, or analyzed results, obtained from the measurements collected by the sensors 3.

The diffused substance can, but not exclusively, have a bactericidal activity.

In particular, the diffused substance can be a molecule causing the parturition or delivery.

The embodiment of FIG. 3 also has such a housing 2 provided with such diffusion means 6, shown by the presence of at least one ejection opening 64.

The specialist in the art will easily design a housing 2 provided with several sets of solenoid valves with their tanks, for example in order to diffuse several different substances. To this end, the housing 2 will advantageously include several openings connected to the solenoid valves.

The embodiment of the device shown in FIG. 3, which advantageously includes a housing 2 comprised of two parts, one 20 including the sensors 3 and the other one 21 including the electronics for processing the measurements performed, also has further particular features.

Indeed, the means for diffusion 6 a substance into the reproductive tract of the female mammal in the form of a solenoid valve 60 are supplemented with a diffusing and/or bactericidal coating 65. This option could of course be preferred and chosen exclusively over the presence of a diffusion system with a solenoid valve, which is more complex.

Turning back to the parameters likely to be used, parameters, other than the temperature or the hormone concentration, could be collected by the housing 2, by means of adapted sensors 3. Being in addition to the temperature or the hormone concentration, these parameters can thus permit to refine the detection.

Depending on the needs or availabilities, the specialist in the art can also select the parameters to be used, the spirit of the invention recommending the recording of at least two different parameters in order to ensure the reliability of the analysis, whether it is automated or not.

Indeed, it will be obvious for the specialist in the art to modulate the sensor composition of the device depending on the functionalities of said selected sensors.

Further parameters that can be used in a relevant way are for example the rate of heartbeat or, in the case of using the device in animals, the frequency of the movement of the legs in movement, or the change in the spectrum of the mooing.

Accordingly, further sensors internal to the housing 2 are thus necessary, such as an acoustic sensor not shown, in particular sensitive to the noise of the heartbeats of both the female mammal and a possible fetus, or to mooing.

A flush pressure sensor, not shown in the figures, also permits to record the rate of the heartbeat.

Such a sensor also permits to detect contractions in the case of imminence of parturition.

In mammals other than the human being, a motion sensor sensitive to the displacements can act as a pedometer in order to evaluate their agitation.

Finally, a camera-type sensor could usefully be used for visualizing and quantifying the dilation of the cervix.

It should be reminded that according to the invention, the same parameters as those measured for detecting a fertile state can advantageously also permit to detect a state of pregnancy, for example following a successful artificial insemination, or also the imminence of a parturition or delivery.

Accordingly and according to a variant of the device according to the invention, the housing 2 can also include an ultrasound generator. Such a generator can indeed be used for obtaining information on a possible fetus.

1. Device for determining the physiological state of female mammals related to procreation, the device comprising:
a sealed housing being inserted into the reproductive tract of a female mammal;
at least two sensors placed on a periphery of said housing and/or internally in said housing, the sensors capturing different physical and/or psychological data; and
power supply means for enabling independent operation, wherein the housing is associated with means for processing data collected by the sensors and means for transmitting the collected data and/or the results obtained by data processing.

2. Device according to claim 1, wherein one of the sensors is comprised of a temperature sensor.

3. Device according to claim 1, wherein one of the sensors is comprised of a sensor for determining hormone concentration.

4. Device according to claim 1, wherein one of the sensors is comprised of a sensor for determining resistance and/or resistivity of the vaginal mucous membrane and/or mucus.

5. Device according to claim 1, wherein one of the sensors is comprised of a sensor sensitive to the estrogens.

6. Device according to claim 1, wherein one of the sensors is comprised of a sensor reacting to the pheromones secreted by Bertholin glands.

7. Device according to claim 1, wherein one of the sensors is comprised of a bioactive coating on the housing.

8. Device according to claim 1, wherein one of the sensors is comprised of an acoustic sensor.

9. Device according to claim 1, wherein one of the sensors is comprised of a pressure sensor.

10. Device according to claim 1, wherein one of the sensors is comprised of a motion sensor.

11. Device according to claim 1, wherein one of the sensors is comprised of a camera-type sensor.

12. Device according to claim 1, wherein the means for processing the data comprises:
at least one memory;
at least one control unit of the memory; and
at least one central processing unit ensuring processing of the data obtained from the sensors.

13. Device according to claim 12, wherein the central processing unit is located inside the housing, comprising a memory and processing software.

14. Device according to claim 1, wherein the means for processing the data comprises:
a signal transmitting-receiving unit inside the housing; and
one external central processing unit, said signal transmitting-receiving unit dialoguing with the central processing unit.

15. Device according to claim 1, wherein the housing further comprises means for diffusing a substance into the reproductive tract of the female mammal.

16. Device according to claim 15, wherein the means for diffusing a substance into the reproductive tract of the female comprises at least one solenoid valve associated to a pressurized tank.

17. Device according to claim 16, wherein the solenoid valve is controlled by a control unit.

18. Device) according to claim 15, wherein the means for diffusing a substance into the reproductive tract of the female mammal comprises at least one diffusion and/or bactericidal coating arranged on a periphery of the housing.

19. Device according to claim 1, wherein the housing further comprises an ultrasound generator.
